(19) United States
(12) Patent Application Publication
(10) Pub. No.: US 2023/0420135 A1
(43) Pub. Date: Dec. 28, 2023

(54) METHOD FOR ASSESSING FASTING STATUS AND FASTING STATUS ASSESSING SYSTEM

(71) Applicant: China Medical University, Taichung City (TW)

(72) Inventors: Chin-Chi Kuo, Taichung City (TW); Che-Chen Lin, Taichung City (TW); Hsiu-Yin Chiang, Taichung City (TW); Min-Yen Wu, Taichung City (TW)

(73) Assignee: China Medical University, Taichung City (TW)

(21) Appl. No.: 18/337,225

(22) Filed: Jun. 19, 2023

Related U.S. Application Data
(60) Provisional application No. 63/355,300, filed on Jun. 24, 2022.

(51) Int. Cl. G16H 50/20; A61B 5/00; G16H 10/60

(52) U.S. Cl. CPC G16H 50/20; G16H 10/60; A61B 5/4866

(57) ABSTRACT

A method for assessing fasting status includes the following steps. A fasting blood glucose database is provided, wherein the fasting blood glucose database includes a plurality of fasting blood glucose data and a plurality of non-fasting blood glucose data. A model establishing step is performed, wherein the plurality of fasting blood glucose data and the plurality of non-fasting blood glucose data are trained to achieve a convergence by a machine-learning model so as to obtain a fasting-status assessing classifier. An ontological data of a subject is provided, wherein the ontological data includes a blood glucose concentration data. An assessing step is performed, wherein the ontological data is analyzed by the fasting-status assessing classifier to obtain an assessing result of fasting status of the subject.

### RELATED APPLICATIONS

This application claims priority to U.S. Provisional Application Ser. No. 63/355,300, filed Jun. 24, 2022, which is herein incorporated by reference.

### BACKGROUND

### Technical Field

The present disclosure relates to a medical information analysis method and a system thereof. More particularly, the present disclosure relates to a method for assessing fasting status and a fasting status assessing system.

### Description of Related Art

With the universal implementation of electronic medical records (EMRs), researchers have actively leveraged real-world EMRs data in diabetes research and management in clinical practice. In current clinical practice, the phenotypes of diabetes mellitus are defined by various combinations of the different components of the EMRs, such as diagnostic codes, medication data and laboratory values related to glucose homeostasis. Thus, how to develop a method to identify the patients with prediabetes and diabetes mellitus with high validity has become increasingly fundamental in improving the quality of care of the patients and preventing complications associated with diabetes mellitus.

In the clinical, there are several commonly used diagnostic criteria for diabetes mellitus, and the most important one is the value of fasting blood glucose level. In detail, the blood sample of the patient is drawn after eight hours of fasting, and under normal conditions, the blood glucose level should be less than 100 mg/dL. However, if the blood glucose level exceeds 126 mg/dL, the patient will be considered to suffer from diabetes mellitus. In recent years, several studies have indicated that the machine learning algorithms may better identify the blood glucose level for diagnosing the diabetic status in the EMRs. However, these studies usually assumed that the fasting blood glucose values are valid if the fasting blood glucose is labeled as such by the clinical laboratory, so that the aforementioned studies may lead to potential overestimation of the fasting status. Further, the information regarding whether the patients have been given instructions to fast before phlebotomy is also not recorded in the EMRs.

Therefore, how to provide a rapid and accurate method to identify whether the patients are under the fasting condition or not so as to facilitate the diagnosis of the diabetes mellitus and to rapidly and accurately formulate an appropriate treatment strategy, has become the goal of the relevant academic and industry development.

### SUMMARY

According to one aspect of the present application, a method for assessing fasting status includes the following steps. A fasting blood glucose database is provided, wherein the fasting blood glucose database includes a plurality of fasting blood glucose data and a plurality of non-fasting blood glucose data. A model establishing step is performed, wherein the plurality of fasting blood glucose data and the plurality of non-fasting blood glucose data are trained to achieve a convergence by a machine-learning model so as to obtain a fasting-status assessing classifier. An ontological data of a subject is provided, wherein the ontological data includes a blood glucose concentration data. An assessing step is performed, wherein the ontological data is analyzed by the fasting-status assessing classifier to obtain an assessing result of fasting status of the subject.

According to another aspect of the present application, a fasting status assessing system includes a non-transitory machine readable medium and a processor. The non-transitory machine readable medium is for storing an ontological data of a subject, wherein the ontological data includes a blood glucose concentration data. The processor is signally connected to the non-transitory machine readable medium and includes a fasting-status assessing classifier, wherein the ontological data is analyzed by the fasting-status assessing classifier to obtain an assessing result of fasting status of the subject.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for assessing fasting status according to one embodiment of the present disclosure.

FIG. 2 is a block diagram of a fasting status assessing system according to another embodiment of the present disclosure.

FIG. 3 shows a density plot of all samples of the fasting blood glucose database that are stratified by the availability of HbA1c concentration data on the same day according to Example 1.

FIG. 4 shows a density plot of the fasting blood glucose data and the non-fasting blood glucose data of the fasting blood glucose database according to Example 1.

FIG. 5 shows a density plot of the fasting blood glucose data and the non-fasting blood glucose data of non-diabetic subjects in the fasting blood glucose database measured on the same day according to Example 1.

FIG. 6 shows a density plot of the fasting blood glucose data and the non-fasting blood glucose data of diabetic subjects in the fasting blood glucose database measured on the same day according to Example 1.

FIG. 7A shows a scatter plot of the HbA1c-derived averaged glucose level data and the fasting blood glucose data of diabetic subjects according to Example 1.

FIG. 7B shows a scatter plot of the HbA1c-derived averaged glucose level data and the fasting blood glucose data of non-diabetic subjects according to Example 1.

FIG. 8 shows discrimination statistics results of different machine-learning models of the fasting status assessing system according to Example 1.

FIG. 9 shows calibration results of different machine-learning models of the fasting status assessing system according to Example 1.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

[Method for Assessing Fasting Status of the Present Disclosure]

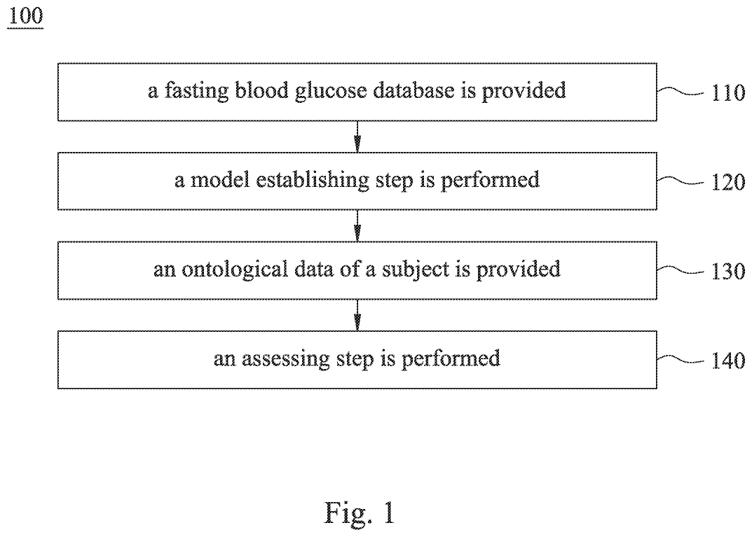

Reference is made to FIG. 1, which is a flow chart of a method 100 for assessing fasting status according to one embodiment of the present disclosure. The method 100 for assessing fasting status of the present disclosure includes Step 110, Step 120, Step 130 and Step 140.

In Step 110, a fasting blood glucose database is provided, wherein the fasting blood glucose database includes a plurality of fasting blood glucose data and a plurality of non-fasting blood glucose data. The fasting blood glucose database can be a dataset obtained from the results of glucose measurements in electronic medical records (EMRs) of subjects in a hospital, and the fasting blood glucose data and the non-fasting blood glucose data used in the present disclosure are further described as follows.

In detail, the fasting blood glucose database can further include a plurality of ontological fasting glucose level data and a plurality of HbA1c-derived averaged glucose level data, and each of the plurality of ontological fasting glucose level data is corresponding to one of the plurality of HbA1c-derived averaged glucose level data.

In the present disclosure, each of the plurality of HbA1c-derived averaged glucose level data is obtained by calculating an HbA1c concentration data corresponding thereto with the following formula:

AG (mg/dL)=28.7×A1C−46.7,

wherein AG is the HbA1c-derived averaged glucose level data, and A1C is the HbA1c concentration data. Each of the plurality of ontological fasting glucose level data is compared to the HbA1c-derived averaged glucose level data corresponding thereto so as to obtain the plurality of fasting blood glucose data. In detail, the HbA1c-derived averaged glucose level data summarizes the daily glucose variation over the past 90 days of a subject and depicts an averaged value between the lowest glucose level and the highest glucose level in this time window among the subject with a stable metabolic state. Thus, if the plurality of fasting blood glucose data are truly obtained in the fasting status, the values thereof will be theoretically less than the values of the HbA1c-derived averaged glucose level data corresponding thereto.

Further, because the most important criteria for diagnosing the diabetes mellitus is the value of the fasting blood glucose level, the ontological fasting glucose level data will be further compared to the HbA1c-derived averaged glucose level data corresponding thereto depending on whether the subject is a diabetic patient or not so as to investigate the “true” fasting status of the fasting blood glucose data. In detail, when the ontological fasting glucose level data is not an ontological fasting glucose level data of a diabetic patient and is classified as the fasting blood glucose data, the ontological fasting glucose level data will satisfy the following conditions: AC_{ontological}<100 mg/dL and A1C<5.5%; or AC_{ontological}<AG−1 standard deviation of AC_{ontological}; wherein AC_{ontological} is the ontological fasting glucose level data, A1C is the HbA1c concentration data, and AG is the HbA1c-derived averaged glucose level data. On the contrary, when the ontological fasting glucose level data is an ontological fasting glucose level data of a diabetic patient and is classified as the fasting blood glucose data, the one of the plurality of ontological fasting glucose level data will satisfy the following condition: AC_{ontological}<AG; wherein AC_{ontological} is the ontological fasting glucose level data, and AG is the HbA1c-derived averaged glucose level data. Once the aforementioned conditions are satisfied, the ontological fasting glucose level data will be classified as the fasting blood glucose data for following analysis.

Furthermore, the ontological fasting glucose level data obtained from EMRs will be classified as non-fasting blood glucose data if at least one of the following conditions is satisfied: (1) the ontological fasting glucose level data are labeled as a post cibum glucose level data or a random glucose level data; (2) the ontological fasting glucose level data includes additional descriptions such as “one-touch”, “bedside check”, “PC (post cibum)”, or contained descriptions indicating active food intake before phlebotomy; and (3) more than one ontological fasting glucose level data is obtained from a single person on the same day, and the earliest obtained ontological fasting glucose level data is considered as a non-fasting blood glucose data.

Therefore, the fasting blood glucose data and the non-fasting blood glucose data of the fasting blood glucose database of the present disclosure have been confirmed before analysis, so that the assessing accuracy of the method 100 for assessing fasting status of the present disclosure can be further enhanced.

In Step 120, a model establishing step is performed, wherein the plurality of fasting blood glucose data and the plurality of non-fasting blood glucose data are trained to achieve a convergence by a machine-learning model so as to obtain a fasting-status assessing classifier. In detail, in the method 100 for assessing fasting status, the fasting-status assessing classifier is established by training the plurality of fasting blood glucose data and the plurality of non-fasting blood glucose data of the fasting blood glucose database, and a ratio between a number of the plurality of fasting blood glucose data and a number of the plurality of non-fasting blood glucose data in the fasting blood glucose database is 1:1 so as to maintain the training accuracy of the machine-learning model. Further, the machine-learning model can be a gradient descent algorithm. Preferably, the gradient descent algorithm can be an XGBoost machine-learning model, a CatBoost machine-learning model or an H2O AutoML ensemble machine-learning model so as to better handle the categorical variables and then output the features for assessing the fasting status of subjects, and the present disclosure is not limited thereto.

In Step 130, an ontological data of a subject is provided, wherein the ontological data includes a blood glucose concentration data. In detail, the subject can be an inpatient or an outpatient, and the ontological data can be obtained from the measurements using the blood samples from inpatient or outpatient services. Further, the subject also can be a diabetic patient or a non-diabetic patient, and the present disclosure is not limited thereto.

In Step 140, an assessing step is performed, wherein the ontological data is analyzed by the fasting-status assessing classifier to obtain an assessing result of fasting status of the subject. The assessing result of fasting status of the subject is for analyzing whether the subject is under the fasting condition or not so as to facilitate the diagnosis of the diabetes mellitus and to rapidly and accurately formulate an appropriate treatment strategy.

Further, the ontological data of the subject can further include a vital signs data, and the vital signs data can include an age data, a gender data, a body mass index data, a medical history data, and a residence-to-location distance data. The blood glucose concentration data, the age data, the gender data, the body mass index data, the medical history data and the residence-to-location distance data of the ontological data are analyzed by the fasting-status assessing classifier in the assessing step so as to output the assessing result of fasting status of the subject. In detail, the fasting status might be associated with the travel time to the healthcare facility, so the residence-to-location distance data is obtained by calculating a straight-line distance or a traffic distance between a residence of the subject and a fasting-status assessing location. Therefore, the assessing accuracy of the method 100 for assessing fasting status of the present disclosure can be further enhanced.

Therefore, by analyzing the ontological data of the subject by the fasting-status assessing classifier, and the fasting-status assessing classifier is established by training the fasting blood glucose data and the non-fasting blood glucose data of the fasting blood glucose database, the method 100 for assessing fasting status of the present disclosure can rapidly and accurately output the assessing result of fasting status of the subject. Hence, it is favorable for the diagnosis of the diabetes mellitus and designing the subsequent medical plans of the subject, and thus the method 100 for assessing fasting status of the present disclosure has excellent clinical application potential.

[Fasting Status Assessing System of the Present Disclosure]

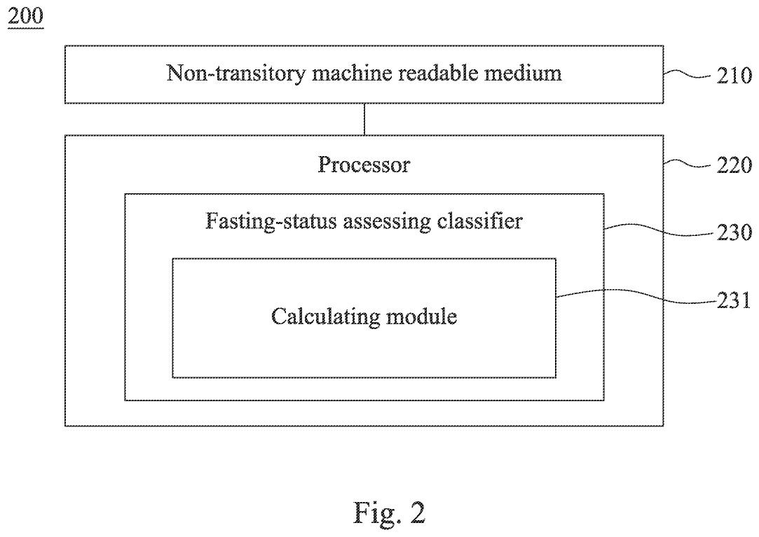

Reference is made to FIG. 2, which is a block diagram of a fasting status assessing system 200 according to another embodiment of the present disclosure. The fasting status assessing system 200 includes a non-transitory machine readable medium 210 and a processor 220.

The non-transitory machine readable medium 210 is for storing an ontological data of a subject, and the ontological data includes a blood glucose concentration data. In detail, the non-transitory machine readable medium 210 can be further for storing a fasting blood glucose database, the fasting blood glucose database includes a plurality of fasting blood glucose data and a plurality of non-fasting blood glucose data, and the plurality of fasting blood glucose data and the plurality of non-fasting blood glucose data are used to establish a fasting-status assessing classifier. Further, the establishing method of the fasting-status assessing classifier is described in Step 120 of the method 100 for assessing fasting status of the present disclosure, so that details thereof will not be described herein again.

The processor 220 is signally connected to the non-transitory machine readable medium 210 and includes a fasting-status assessing classifier 230, wherein the ontological data of the subject is analyzed by the fasting-status assessing classifier 230 to obtain an assessing result of fasting status of the subject. The assessing result of fasting status of the subject is for analyzing whether the subject is under the fasting condition or not so as to facilitate the diagnosis of the diabetes mellitus and to rapidly and accurately formulate an appropriate treatment strategy.

Further, in the fasting status assessing system 200 of the present disclosure, the fasting-status assessing classifier 230 can include a calculating module 231, the fasting blood glucose database can further include a plurality of ontological fasting glucose level data and a plurality of HbA1c-derived averaged glucose level data, each of the plurality of ontological fasting glucose level data is corresponding to one of the plurality of HbA1c-derived averaged glucose level data, and each of the plurality of ontological fasting glucose level data is compared to the HbA1c-derived averaged glucose level data corresponding thereto by the calculating module 231 so as to obtain the plurality of fasting blood glucose data. Further, the definitions of the fasting blood glucose data and the non-fasting blood glucose data of the present disclosure and the calculating details of the HbA1c-derived averaged glucose level data are the same to that of the method 100 for assessing fasting status of the present disclosure, so that details thereof will not be described herein again.

Therefore, by analyzing the ontological data of the subject by the fasting-status assessing classifier 230 of the present disclosure, the fasting status assessing system 200 of the present disclosure can rapidly and accurately output the assessing result of fasting status of the subject for designing the subsequent medical plans of the subject, and thus the fasting status assessing system 200 of the present disclosure has excellent clinical application potential.

### Example 1

I. Fasting Blood Glucose Database

In Example 1, the fasting blood glucose database is collected by China Medical University Hospital, and the clinical research study is approved by China Medical University & Hospital Research Ethics Committee, which is numbered as CMUH105-REC3-068. The fasting blood glucose database includes the EMRs of 2,873,887 subjects in the outpatient that are aged of 59.7±14.6 years old and collected during 2003 to 2008, wherein 945,792 of the subjects are underwent ante cibum glucose measurements using sera samples, and a total of 2,196,833 of ontological fasting glucose level data are obtained. Further, 784,340 of the ontological fasting glucose level data (149,654 of the subjects) are respectively simultaneously provided with at least one HbA1c concentration data, wherein the plurality of ontological fasting glucose level data are determined by using the Beckman Oxygen electrode (glucose oxidase method) with Beckman Synchron® LX20 and Beckman UniCel® DxC 800 (Beckman Coulter Inc., Brea, CA, USA), and the HbA1c concentration data is measured by using the boronate affinity and high-performance liquid chromatography (HPLC) methods with Primus CLC385 analyzer, the cation exchange HPLC methods with Tosoh HLC-723 G7 (Tosoh Corporation, Tokyo, Japan), and the boronate affinity and HPLC methods with Trinity Biotech Premier Hb9210.

Further, each of the 784,340 ontological data also includes a vital signs data, and the vital signs data can include an age data, a gender data, a body mass index data, a medical history data and a residence-to-location distance data, wherein the medical history data includes the disease histories of diabetes mellitus, coronary artery disease, myocardial infarction, stroke or congestive heart failure or other cardiovascular diseases. The presence of hypertension or type 2 diabetes mellitus is captured based on associated ICD-9/-10 codes or the use of glucose-lowering medications or antihypertensive agents. Further, the medication records, the health care provider specialty and the biochemical measures are also included in the ontological data. Furthermore, the residence-to-location distance data is a straight-line distance or a traffic distance between a residence of the subject and a fasting-status assessing location, and the residence-to-location distance data can be calculated in two steps. First, a geocoding application programming interface developed by Google Maps is used to transform the map coordinates of the residence of the subject and the fasting-status assessing location. Then, the distance between the residence of the subject and the fasting-status assessing location is calculated by using the geographic information system (ArcGIS version 10; ESRI, Redlands, CA, USA).

II. Data Preprocessing

1. Determining of Fasting Blood Glucose Data

In the present disclosure, the fasting blood glucose database further includes a plurality of HbA1c-derived averaged glucose level data, wherein each of the plurality of HbA1c-derived averaged glucose level data is obtained by calculating an HbA1c concentration data corresponding thereto with the following formula:

AG (mg/dL)=28.7×A1C−46.7,

wherein AG is the HbA1c-derived averaged glucose level data, and A1C is the HbA1c concentration data.

In the present disclosure, the HbA1c-derived averaged glucose level data is used to determine the “true” ontological fasting status on blood glucose measurements. In detail, each of the plurality of ontological fasting glucose level data is corresponding to one of the plurality of HbA1c-derived averaged glucose level data, and each of the plurality of ontological fasting glucose level data is compared to the HbA1c-derived averaged glucose level data corresponding thereto so as to obtain a plurality of fasting blood glucose data.

In more detail, the ontological fasting glucose level data will be classified as fasting blood glucose data according to the disease history of diabetes mellitus of the subject. When the subject is not a diabetic patient, the ontological fasting glucose level data of the subject will satisfy the following conditions: AC_{ontological}<100 mg/dL and A1C<5.5%; or AC_{ontological}<AG−1 standard deviation of AC_{ontological}; and when the subject is a diabetic patient, the ontological fasting glucose level data will satisfy the following condition: AC_{ontological}<AG. As shown above, AC_{ontological} is the ontological fasting glucose level data, A1C is the HbA1c concentration data, and AG is the HbA1c-derived averaged glucose level data. Once the aforementioned conditions are satisfied, the ontological fasting glucose level data will be classified as the fasting blood glucose data.

2. Determining of Non-Fasting Blood Glucose Data

The ontological fasting glucose level data will be classified as non-fasting blood glucose data if at least one of the following conditions are satisfied: (1) the ontological fasting glucose level data are labeled as a post cibum glucose level data or a random glucose level data; (2) the ontological fasting glucose level data includes additional descriptions such as “one-touch”, “bedside check”, or “PC (post cibum)” or contained descriptions indicating active food intake before phlebotomy; and (3) more than one ontological fasting glucose level data is obtained from a single person on the same day, and the earliest obtained ontological fasting glucose level data is considered as a non-fasting blood glucose data.

Finally, 604,639 fasting blood glucose data (133,296 of the subjects) and 179,701 non-fasting blood glucose data (55,326 of the subjects) are obtained to establish the fasting-status assessing classifier of Example 1.

III. Establishing Fasting-Status Assessing Classifier

In the establishing process of the fasting-status assessing classifier of Example 1, a ratio between a number of the plurality of fasting blood glucose data and a number of the plurality of non-fasting blood glucose data in the fasting blood glucose database is further adjusted to 1:1 so as to balance the weight of the fasting blood glucose data and the non-fasting blood glucose data and then enhance the assessing accuracy of the fasting-status assessing classifier. Thus, the number of the fasting blood glucose data is adjusted to 179,701, which is the same to that of the non-fasting blood glucose data (a total of 359,402 ontological fasting glucose level data of 93,958 subjects).

Further, the subjects are separated into a training dataset and a testing dataset at an 80/20 proportion, wherein 143,355 fasting blood glucose data and 143,306 non-fasting blood glucose data (a total of 75,160 subjects) are included in the training dataset, and 36,346 fasting blood glucose data and 36,395 non-fasting blood glucose data (a total of 18,798 subjects) are included in the testing dataset. Additionally, the method of cluster analysis is used to deal with the problem of complementing missing values. Then, the plurality of fasting blood glucose data and the plurality of non-fasting blood glucose data are trained to achieve a convergence by a machine-learning model so as to obtain the fasting-status assessing classifier of Example 1 of the present disclosure, wherein the machine-learning model can be a gradient descent algorithm, and the a gradient descent algorithm can be a XGBoost machine-learning model, a CatBoost machine-learning model or an H2O AutoML ensemble machine-learning model so as to better handle the categorical variables and explore the predictive performance using multiple machine-learning models. Further, a multiple logistic regression model is used to analyze the training dataset and the testing dataset as a comparative example so as to further illustrate the assessing efficacy of the fasting-status assessing classifier of the present disclosure.

In Example 1, the hyperparameters of the XGBoost machine-learning model are determined by using the Tree of Parzen Estimators (TPE) method. Taking the implementation of the XGBoost machine-learning model in Python as an example, the finalized hyperparameters are set as tree depth=8, the learning rate=0.1, the gamma=0.5, the minimum sum of instance weight=7, the number of estimators=300, and the remaining parameters are set by using the default setting. Detailed parameter ranges for grid research of the XGBoost machine-learning model and the CatBoost machine-learning model in Example 1 are shown in Table 1.

TABLE 1

| Machine-learning model | XGBoost | CatBoost |
|---|---|---|
| Max depth | 3-8 | 5-10 |
| Learning rate | 0.01-0.1 | 0.001-0.1 |
| Number of estimators | 50-300 | 20-300 |
| Bagging temperature | — | 3-10 |
| Gamma | 0.5-2.0 | — |
| Subsample | 0.6-1.0 | — |
| Colsample by tree | 0.6-1.0 | — |

Further, the performance quantification of the fasting-status assessing classifiers established by different machine-learning models are evaluated in terms of area under the receiver operating characteristic curve (“AUROC” hereafter), accuracy, precision, recall, and F1-score using a 5-fold cross-validation scheme, and the bootstrapping method with 2000 repetitions is used to statistically test the difference between the paired AUROCs. All statistical analyses are performed by using SAS version 9.4 (SAS Institute Inc., Cary, NC, USA), R version 3.5.1 (R Foundation for Statistical Computing, Vienna, Austria), and Python version 3.7.3 under a Linux operating system. The Python package version is 1.5.2 for the XGBoost machine-learning model, 1.0.4 for the CatBoost machine-learning model, 3.36.0.2 for the H2O AutoML ensemble machine-learning model, and the two-sided statistical significance level is set at 0.05.

IV. Results

1. Distribution of Glucose Level by Fasting and Diabetic Status

Reference is made to Table 2, which shows a comparison of demographic and biochemical profiles of all the ontological fasting glucose level data used to establish the fasting-status assessing classifier according to diabetes mellitus and theoretical fasting status.

TABLE 2

| | Non-diabetes mellitus | | | | Diabetes mellitus | | | |
|---|---|---|---|---|---|---|---|---|
| | | | Non- | | | | Non- | |
| | Overall | Fasting | fasting | p | Overall | Fasting | fasting | p |
| Number | 118383 | 53080 | 65 303 | | 241019 | 126621 | 114398 | |
| Age (years old) | 55.3 | 50.7 | 59.1 | <0.01 | 61.9 | 62.6 | 61.1 | <0.01 |
| | (15.0) | (15.6) | (13.4) | | (13.9) | (13.3) | (14.6) | |
| Male | 66333 | 29147 | 37186 | <0.01 | 127301 | 66791 | 60510 | 0.47 |
| | (56.0) | (54.9) | (56.9) | | (52.8) | (52.8) | (52.9) | |
| BMI (kg/m²) | 25.4 | 24.8 | 26.1 | <0.01 | 26.1 | 26.2 | 25.9 | <0.01 |
| | (4.85) | (4.83) | (4.78) | | (4.63) | (4.65) | (4.60) | |
| Sampling timing | — | — | — | <0.01 | — | — | — | <0.01 |
| of the day | | | | | | | | |
| 7:00-12:59 | 109784 | 48168 | 61616 | — | 225770 | 119210 | 106560 | — |
| | (92.7) | (90.8) | (94.4) | | (96.7) | (94.2) | (93.2) | |
| 13:00-17:59 | 7465 | 4322 | 3143 | — | 11761 | 5805 | 5956 | — |
| | (6.31) | (8.14) | (4.81) | | (4.88) | (4.58) | (5.21) | |
| 18:00-22:59 | 1134 | 590 | 544 | — | 3488 | 1606 | 1882 | — |
| | (0.96) | (1.11) | (0.83) | | (1.45) | (1.27) | (1.65) | |
| Interval between | 32.0 | 24.9 | 37.8 | <0.01 | 57.6 | 58.71 | 56.39 | <0.01 |
| request and sampling | (40.8) | (37.9) | (42.2) | | (34.2) | (33.3) | (35.2) | |
| day (day) | | | | | | | | |
| No. of outpatient visits | 9.42 | 7.44 | 11.0 | <0.01 | 19.56 | 19.07 | 20.11 | <0.01 |
| | (17.6) | (15.8) | (18.7) | | (24.7) | (23.7) | (25.8) | |
| Residence-to-location | 15.4 | 17.7 | 13.6 | <0.01 | 10.6 | 10.6 | 10.59 | 0.87 |
| distance (km) | (76.3) | (89.7) | (63.9) | | (55.4) | (51.7) | (59.2) | |
| Concomitant lipid | 95750 | 47735 | 48015 | <0.01 | 147021 | 81320 | 65701 | <0.01 |
| testing | (80.9) | (89.9) | (73.5) | | (61.0) | (64.2) | (57.4) | |
| Division | — | — | — | <0.01 | — | — | — | <0.01 |
| General Medicine | 19268 | 6913 | 12355 | — | 19988 | 10277 | 9711 | — |
| | (16.3) | (13.02) | (18.92) | | (8.29) | (8.12) | (8.49) | |
| Metabolism/ | 14190 | 3553 | 10637 | — | 132971 | 70496 | 62475 | — |
| Endocrinology | (12.0) | (6.69) | (16.29) | | (55.2) | (55.67) | (54.61) | |
| Nephrology | 7461 | 2070 | 5391 | — | 21015 | 10298 | 10717 | — |
| | (6.30) | (3.9) | (8.26) | | (8.72) | (8.13) | (9.37) | |
| Cardiology | 20837 | 6260 | 14577 | — | 36060 | 20265 | 15795 | — |
| | (17.6) | (11.79) | (22.32) | | (15.0) | (16) | (13.81) | |
| Family Medicine | 10810 | 4708 | 6102 | — | 16022 | 8243 | 7779 | — |
| | (9.13) | (8.87) | (9.34) | | (6.65) | (6.51) | (6.8) | |
| Health management | 34572 | 25217 | 9355 | — | 766 | 458 | 308 | — |
| center | (29.2) | (47.51) | (14.33) | | (0.32) | (0.36) | (0.27) | |
| Surgery | 5469 | 2782 | 2687 | — | 2915 | 1552 | 1363 | — |
| | (4.62) | (5.24) | (4.11) | | (1.21) | (1.23) | (1.19) | |
| Pediatrics | 1109 | 710 | 399 | — | 3607 | 1065 | 2542 | — |
| | (0.94) | (1.34) | (0.61) | | (1.50) | (0.84) | (2.22) | |
| Chinese medicine | 4232 | 735 | 3497 | — | 7226 | 3753 | 3473 | — |
| | (3.57) | (1.38) | (5.36) | | (3.00) | (2.96) | (3.04) | |
| Other | 435 | 132 | 303 | — | 449 | 214 | 235 | — |
| | (0.37) | (0.25) | (0.46) | | (0.19) | (0.17) | (0.21) | |
| Comorbidity | — | — | — | — | — | — | — | — |
| Hypertension | 33868 | 10916 | 22952 | <0.01 | 143831 | 76406 | 67425 | <0.01 |
| | (28.6) | (20.6) | (35.2) | | (59.7) | (60.34) | (58.94) | |
| Coronary artery | 12906 | 3981 | 8925 | <0.01 | 46568 | 24982 | 21586 | <0.01 |
| disease | (10.9) | (7.50) | (13.7) | | (19.3) | (19.73) | (18.87) | |
| Stroke | 8713 | 3236 | 5477 | <0.01 | 31375 | 16602 | 14773 | 0.15 |
| | (7.36) | (6.10) | (8.39) | | (13.0) | (13.11) | (12.91) | |
| Biochemical variables | — | — | — | — | — | — | — | — |
| Glucose (mg/dL) | 124 | 96.9 | 145 | <0.01 | 163 | 132 | 197 | <0.01 |
| | (47.6) | (19.5) | (52.3) | | (62.5) | (36.7) | (67.1) | |
| HbA1c (%) | 6.48 | 5.86 | 6.97 | <0.01 | 7.55 | 7.59 | 7.50 | <0.01 |
| | (1.44) | (0.98) | (1.57) | | (1.50) | (1.42) | (1.58) | |
| Hemoglobin (g/dL) | 14.0 | 14.1 | 13.7 | <0.01 | 11.8 | 12.0 | 11.6 | <0.01 |
| | (1.97) | (1.77) | (2.23) | | (2.29) | (2.21) | (2.35) | |
| Total cholesterol | 191 | 193 | 190 | <0.01 | 177 | 174 | 180 | <0.01 |
| (mg/dL) | (41.5) | (39.4) | (43.6) | | (43.5) | (41.6) | (45.5) | |
| LDL (mg/dL) | 114 | 116 | 111 | <0.01 | 97.2 | 96.6 | 97.9 | <0.01 |
| | (34.6) | (33.7) | (35.4) | | (33.0) | (32.2) | (33.9) | |
| HDL (mg/dL) | 47.8 | 49.9 | 45.2 | <0.01 | 43.6 | 43.6 | 43.6 | 0.95 |
| | (13.7) | (14.1) | (12.6) | | (12.5) | (12.2) | (12.8) | |
| Triglyceride (mg/dL) | 148 | 125 | 172 | <0.01 | 179 | 160 | 200 | <0.01 |
| | (168) | (114) | (208) | | (231) | (191) | (269) | |
| BUN (mg/dL) | 14.6 | 12.3 | 18.2 | <0.01 | 33.0 | 31.0 | 35.0 | <0.01 |
| | (12.7) | (8.83) | (16.3) | | (24.3) | (23.2) | (25.3) | |
| Serum creatinine | 1.14 | 1.00 | 1.28 | <0.01 | 1.63 | 1.53 | 1.76 | <0.01 |
| (mg/dL) | (1.51) | (1.09) | (1.82) | | (2.20) | (1.99) | (2.41) | |
| Serum sodium | 139 | 140 | 138 | <0.01 | 137 | 138 | 136 | <0.01 |
| (mmol/L) | (3.61) | (3.22) | (3.86) | | (3.83) | (3.41) | (4.06) | |
| Serum potassium | 4.09 | 4.02 | 4.15 | <0.01 | 4.33 | 4.34 | 4.32 | <0.01 |
| (mmol/L) | (0.52) | (0.49) | (0.54) | | (0.61) | (0.58) | (0.64) | |
| AST (IU/L) | 30.5 | 27.4 | 34.9 | <0.01 | 33.1 | 32.1 | 34.1 | <0.01 |
| | (26.0) | (19.2) | (32.8) | | (29.1) | (25.6) | (32.1) | |
| ALT (IU/L) | 33.1 | 30.0 | 36.4 | <0.01 | 31.1 | 30.4 | 31.9 | <0.01 |
| | (33.1) | (29.6) | (36.0) | | (28.7) | (26.6) | (30.8) | |
| Uric acid (mg/dL) | 6.00 | 5.89 | 6.13 | <0.01 | 6.29 | 6.26 | 6.32 | <0.01 |
| | (1.59) | (1.55) | (1.63) | | (1.80) | (1.78) | (1.83) | |
| Albumin (g/dL) | 4.50 | 4.55 | 4.41 | <0.01 | 3.97 | 4.03 | 3.92 | <0.01 |
| | (0.41) | (0.35) | (0.48) | | (0.54) | (0.50) | (0.56) | |
| Estimated blood | 291 | 290 | 293 | <0.01 | 296 | 294 | 297 | <0.01 |
| osmolality | (7.90) | (7.02) | (8.68) | | (10.1) | (9.48) | (10.5) | |
| Urine specific gravity | 1.02 | 1.02 | 1.02 | 0.13 | 1.02 | 1.02 | 1.02 | <0.01 |
| | (0.01) | (0.01) | (0.01) | | (0.01) | (0.01) | (0.01) | |
| Urine pH | 6.01 | 6.03 | 5.98 | <0.01 | 6.01 | 6.03 | 5.99 | <0.01 |
| | (0.68) | (0.70) | (0.66) | | (0.63) | (0.65) | (0.62) | |

In Table 2, “BMI” refers to the body mass index, “LDL” refers to the low-density lipoprotein, “HDL” refers to the high-density lipoprotein, “BUN” refers to the blood urea nitrogen, “AST” refers to the aspartate aminotransferase, and “ALT” refers to the alanine transaminase. If the definitions of the parameters shown in the following tables are the same to that of Table 2, the details thereof will not be described again. As shown in Table 2, approximately half of the sample population is female (46.1%), and within the 93,958 subjects, 29.2% have been diagnosed with diabetes mellitus. Blood glucose measurements considered to be collected during fasting status are observed in younger non-diabetes subjects but not among younger subjects with diabetes mellitus. Moreover, samples are more likely to be fasting measures if the lipid profiles of the patients are concomitantly examined, and the statistical differences are observed for the majority of the biochemical measures between the fasting blood glucose data and non-fasting blood glucose data. Specifically, levels of triglyceride demonstrate clinically significantly different results (>15 mg/dL) between the fasting blood glucose data and non-fasting blood glucose data, regardless of the diabetic status.

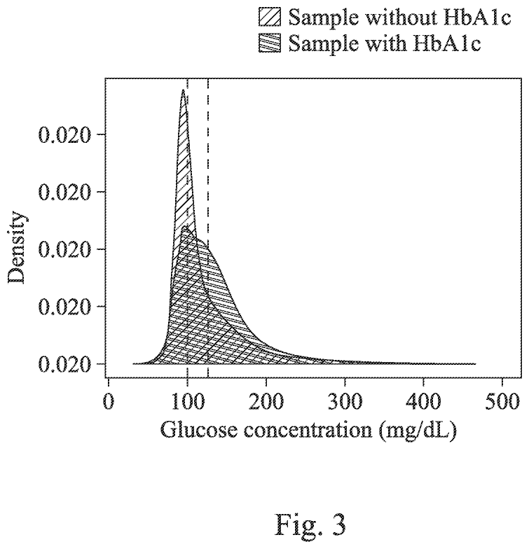

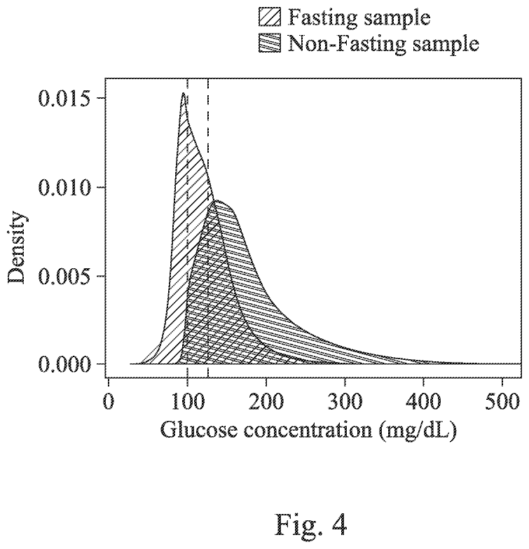

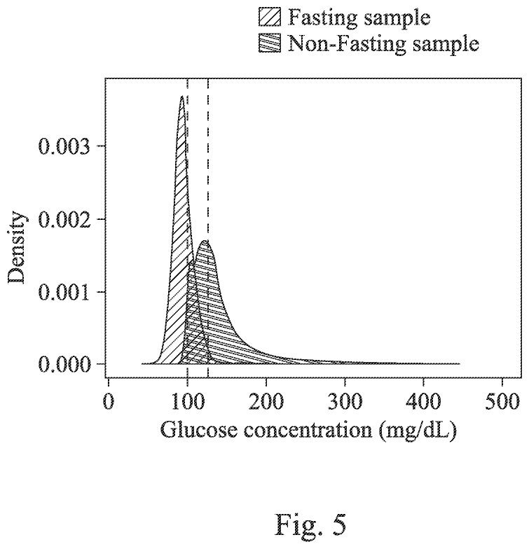

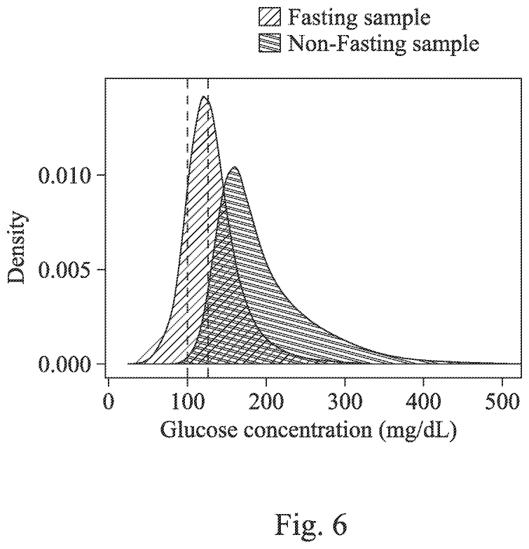

Reference is further made to FIG. 3 to FIG. 6, wherein FIG. 3 shows a density plot of all samples of the fasting blood glucose database that are stratified by the availability of HbA1c concentration data on the same day according to Example 1, FIG. 4 shows a density plot of the fasting blood glucose data and the non-fasting blood glucose data of the fasting blood glucose database according to Example 1, FIG. 5 shows a density plot of the fasting blood glucose data and the non-fasting blood glucose data of non-diabetic subjects in the fasting blood glucose database measured on the same day according to Example 1, and FIG. 6 shows a density plot of the fasting blood glucose data and the non-fasting blood glucose data of diabetic subjects in the fasting blood glucose database measured on the same day according to Example 1.

As shown in FIG. 3, the peak of the density curve of the ontological fasting glucose level data with the HbA1c concentration data (“Sample with HbA1c” in FIG. 3) and the peak of the density curve of the ontological fasting glucose level data without the HbA1c concentration data (“Sample without HbA1c” in FIG. 3) measured in the same day are similar at approximately 100 mg/dL. However, the width of the distribution of the ontological fasting glucose level data with the HbA1c concentration data is wider than that without the HbA1c concentration data.

As shown in FIG. 4, the peak of the density curve of the fasting blood glucose data (“Fasting sample” in FIGS. 4 to 6) and the peak of the density curve of the non-fasting blood glucose data (“Non-Fasting sample” in FIGS. 4 to 6) are separated with peak values slightly lower than 100 and slightly above 126 mg/dL, respectively.

Further, as shown in FIG. 5, among the subjects without diabetes mellitus, a peak shift to the left to <126 mg/dL is noted in the non-fasting blood glucose data compared to the entire fasting blood glucose database. By contrast, as shown in FIG. 6, among the subjects with diabetes mellitus, the peak of the fasting blood glucose data shifts right to approximately 126 mg/dL.

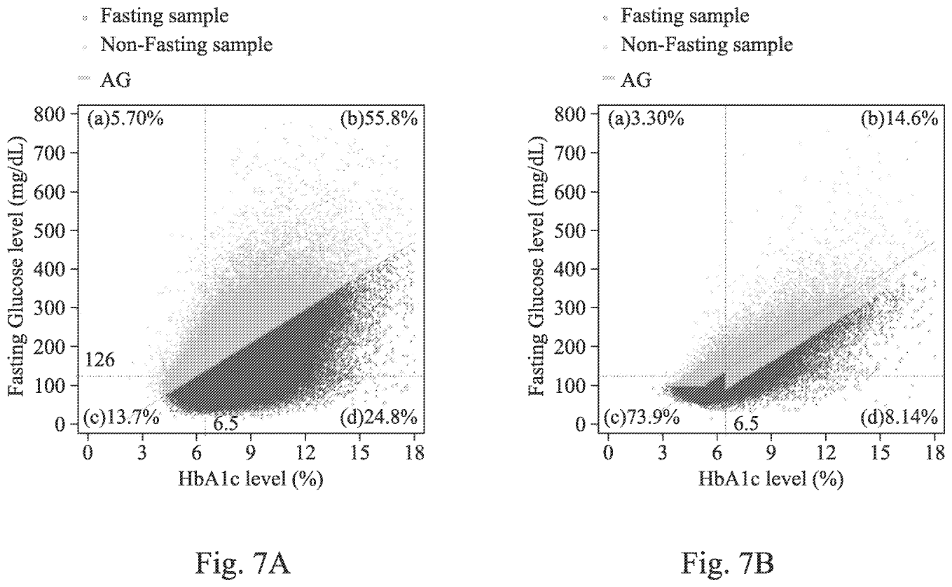

Reference is further made to FIG. 7A and FIG. 7B, wherein FIG. 7A shows a scatter plot of the HbA1c-derived averaged glucose level data and the fasting blood glucose data of diabetic subjects according to Example 1, and FIG. 7B shows a scatter plot of the HbA1c-derived averaged glucose level data and the fasting blood glucose data of non-diabetic subjects according to Example 1. In FIG. 7A and FIG. 7B, “Fasting sample” refers to the fasting blood glucose data, “Non-fasting sample” refers to the non-fasting blood glucose data, and “AG” refers to the HbA1c-derived averaged glucose level data. As shown in FIGS. 7A and 7B, the scatter plots thereof are respectively divided into four quadrants namely (a), (b), (c) and (d) according to the diagnostic criteria of the American Diabetes Association (ADA) by diabetic status, wherein the fasting glucose level is higher than 126 mg/dL and the HbA1c-derived averaged glucose level (“HbA1c level” in FIGS. 7A and 7B) is higher than 6.5% in the zone (b). The amount of the fasting blood glucose data and the non-fasting blood glucose data of the diabetic subjects in the zone (b) (55.8%) is larger than that of the non-diabetic subjects (14.6%).

2. Factors Associated with Non-Fasting Status

Reference is made to Table 3 and Table 4, wherein Table 3 shows the 67 features selected in XGBoost machine-learning model for the classification of fasting status and non-fasting status, and Table 4 shows the odds ratios (95% confidence intervals) of being in the non-fasting status using the ontological fasting glucose level data in the training dataset.

TABLE 3

| Categories of features | Description |
|---|---|
| Demographics | Age, Sex, Residence-to-location distance, Height, |
| | Weight, Body mass index |
| Comorbidity | Hypertension, Diabetes mellitus, Coronary artery |
| | disease, Heart failure, Peripheral vascular disease, |
| | Stroke |
| Medication | Cardiovascular system |
| | Nitroglycerin, Nitrocontin, Isosorbide dinitrate, |
| | Isosorbide-5-mononitrate, Angiotensin-converting |
| | enzyme inhibitors, Angiotensin receptor blockers, |
| | Calcium channel blocker, Hydralazine, Diuretics, |
| | Alpha/Beta blocker, Alpha-1 blocker, Alpha-2 |
| | agonist, Beta blocker, Aspirin, Dipyridamole, |
| | Clopidogrel, Ticlopidine, Ticagrelor, Cilostazol, |
| | Wafarin, Non-vitamin k antagonist |
| | oral-anticoagulant, Heparin |
| | Anti-diabetic agents |
| | Insulin, Metformin, Sulfonylurea, Thiazolinedione, |
| | Alpha glucosidase inhibitors, Dipeptidyl peptidase-4 |
| | inhibitor, Glinide, Sodium-glucose co-transporter 2 |
| | inhibitor |
| | Lipid-lowering agents |
| | Fibrates, Statins |
| Healthcare utilization | The clinical division where the glucose test is |
| | requested, No. of outpatient visits |
| Blood test | Glucose, Hemoglobin, Red blood cell count, Total |
| | cholesterol, HDL, LDL, Triglyceride, BUN, SCr, |
| | Serum sodium, Serum potassium, Albumin, AST, |
| | ALT, Uric acid |
| Blood test time | Sampling timing of the day, Interval between request |
| | and sampling |
| Urinalysis | Urine pH, Urine specific gravity |
| Data augmentation | Estimated blood osmolality, BUN/SCr, AST/ALT, |
| | Hemoglobin/red blood cell count, Serum |
| | sodium/serum potassium |

TABLE 4

| | Univariate analysis | | Model 1 | | Model 2 | |
|---|---|---|---|---|---|---|
| | OR | p- | OR | p- | OR | p- |
| | (95% Cl) | value | (95% Cl) | value | (95% Cl) | value |
| Glucose | 1.20 | <0.001 | 1.16 (1.16-1.16) | <0.001 | 1.23 (1.23-1.23) | <0.001 |
| (5 mg/dL) | (1.20-1.21) | | | | | |
| Age | 1.16 | <0.001 | 1.01 (1.00-1.01) | <0.001 | 1.05 (1.04-1.05) | <0.001 |
| (per 5 years) | (1.16-1.17) | | | | | |
| Male | 1.05 | <0.001 | 1.09 (1.07-1.11) | <0.001 | 1.16 (1.14-1.18) | <0.001 |
| | (1.03-1.06) | | | | | |
| Timing of the day | — | — | — | — | — | — |
| 7:00-12:59 | ref | — | ref | — | ref | — |
| 13:00-17:59 | 0.96 | 0.03 | 0.90 (0.86-0.94) | <0.001 | 0.87 (0.83-0.91) | <0.001 |
| | (0.93-1.00) | | | | | |
| 18:00-22:59 | 1.14 | <0.001 | 0.76 (0.70-0.84) | <0.001 | 0.78 (0.70-0.86) | <0.001 |
| | (1.07-1.22) | | | | | |
| Interval between | 1.07 | <0.001 | 0.95 (0.94-0.96) | <0.001 | 1.08 (1.07-1.08) | <0.001 |
| request and sampling | (1.05-1.08) | | | | | |
| (per 28 days) | | | | | | |
| No. of outpatient visits | 1.03 | <0.001 | 0.99 (0.99-1.00) | <0.001 | 1.02 (1.02-1.02) | <0.001 |
| (per 4 visits) | (1.02-1.04) | | | | | |
| Residence-to-location | 0.998 | 0.04 | 0.998 | 0.03 | 0.998 | 0.01 |
| distance | (0.996- | | (0.997-1.000) | | (0.996-1.00) | |
| (per 10 kms) | 1.000) | | | | | |
| Division | — | — | — | — | — | — |
| Health management | Ref | — | Ref | — | Ref | — |
| center | | | | | | |
| General Medicine | 3.39 | <0.001 | 1.49 (1.43-1.56) | <0.001 | 2.24 (2.14-2.35) | <0.001 |
| | (3.27-3.52) | | | | | |
| Metabolism/ | 2.57 | <0.001 | 0.67 (0.65-0.70) | <0.001 | 2.11 (2.02-2.21) | <0.001 |
| Endocrinology | (2.49-2.65) | | | | | |
| Nephrology | 3.45 | <0.001 | 1.19 (1.13-1.25) | <0.001 | 2.38 (2.25-2.51) | <0.001 |
| | (3.32-3.58) | | | | | |
| Cardiology | 3.03 | <0.001 | 1.09 (1.04-1.14) | <0.001 | 1.99 (1.89-2.08) | <0.001 |
| | (2.93-3.13) | | | | | |
| Family Medicine | 2.80 | <0.001 | 1.03 (0.98-1.08) | 0.22 | 1.90 (1.81-2.00) | <0.001 |
| | (2.69-2.91) | | | | | |
| Surgery | 2.57 | <0.001 | 1.30 (1.22-1.39) | <0.001 | 1.74 (1.62-1.87) | <0.001 |
| | (2.43-2.72) | | | | | |
| Pediatrics | 4.43 | <0.001 | 0.67 (0.60-0.75) | <0.001 | 1.78 (1.59-1.99) | <0.001 |
| | (4.13-4.77) | | | | | |
| Chinese Medicine | 4.07 | <0.001 | 1.00 (0.94-1.06) | 0.92 | 1.86 (1.74-1.99) | <0.001 |
| | (3.87-4.28) | | | | | |
| Other | 3.98 | <0.001 | 1.30 (1.09-1.56) | 0.004 | 1.95 (1.60-2.37) | <0.001 |
| | (3.41-4.64) | | | | | |
| Hypertension | 1.04 | <0.001 | — | — | 1.02 (1.00-1.05) | 0.03 |
| | (1.03-1.06) | | | | | |
| Diabetes mellitus | 0.70 | <0.001 | — | — | 0.09 (0.09-0.09) | <0.001 |
| | (0.68-0.71) | | | | | |
| Coronary artery | 1.07 | <0.001 | — | — | 0.94 (0.91-0.96) | <0.001 |
| disease | (1.05-1.09) | | | | | |
| Stroke | 1.02 | 0.12 | — | — | 0.99 (0.96-1.02) | 0.64 |
| | (0.99-1.04) | | | | | |
| Statin use | 0.78 | <0.001 | — | — | 0.82 (0.80-0.83) | <0.001 |
| | (0.77-0.80) | | | | | |
| Concomitant lipid | 0.69 | <0.001 | — | — | 0.78 (0.76-0.80) | <0.001 |
| profile test | (0.68-0.70) | | | | | |
| AIC | — | — | 297 987 | | 262 835 | |
| AUROC | — | — | 0.820 (0.819-0.822) | | 0.867 (0.866-0.869) | |
| p-value for AUROC | — | — | ref | | <0.001 | |
| difference | | | | | | |

In Table 4, Model 1 is full modeling by all of the 67 features of Example 1, and Model 2 is a parsimonious model that uses the features including glucose, age, male, timing of the day, interval between request and sampling, No. of outpatient visits, residence-to-location distance, division, hypertension, diabetes, coronary artery disease, stroke, statin use, and concomitant lipid testing to assessing the fasting status in Example 1.

As shown in Table 3 and Table 4, the age, the gender, the residence-to-location distance, the timing of blood sampling and the cumulative No. of outpatient visits one year prior to the blood sampling are associated with a higher probability of being in the non-fasting status, and the subjects with the histories of diabetes mellitus, hypertension or coronary artery disease, statin medication, and concomitant lipid and glucose testing are significantly associated with the fasting status. Further, comparing to the odds of non-fasting status among the subjects who visited the health management center, those who are ordered glucose measurement in the departments of metabolism and endocrinology, general medicine, and nephrology are twice as likely to be in non-fasting status (Table 4), and the subjects who underwent concomitant glucose and lipid testing are more likely to follow the fasting instruction, with the odds ratio of being in a non-fasting status of 0.78 (95% CI=0.76-0.80).

3. Machine Learning Performance in Fasting Status Identification

Reference is made to Table 5. Table 5 shows the comparison of performance of determining fasting status by the XGBoost machine-learning model, the CatBoost machine-learning model, the H2O AutoML ensemble machine-learning model and the multiple logistic regression model in the testing dataset of Example 1.

TABLE 5
Parsimonious modeling

| Algorithm/ | | | | | | | |
|---|---|---|---|---|---|---|---|
| Modeling | | | | | | | |
| strategy | Feature | Sensitivity | Specificity | Precision | F1-score | Accuracy | AUROC |
| Logistic | Model 2 | 0.7608 | 0.8084 | 0.8081 | 0.7804 | 0.7845 | 0.868 |
| regression | | | | | | | (0.865-0.870) |
| XGBoost | Model 2 | 0.8261 | 0.7700 | 0.7844 | 0.8047 | 0.7982 | 0.887 |
| | | | | | | | (0.885-0.890) |
| CatBoost | Model 2 | 0.8415 | 0.7614 | 0.7813 | 0.8103 | 0.8017 | 0.889 |
| | | | | | | | (0.887-0.892) |
| H2O | Model 2 | 0.8823 | 0.7093 | 0.7546 | 0.8135 | 0.7964 | 0.886 |
| Ensemble | | | | | | | (0.884-0.889) |

Full modeling

| Algorithm/ | | | | | | | |
|---|---|---|---|---|---|---|---|
| Modeling | | | | | | | |
| strategy | Feature | Sensitivity | Specificity | Precision | F1-score | Accuracy | AUROC |
| XGBoost | 67 | 0.8394 | 0.7785 | 0.7934 | 0.8158 | 0.8092 | 0.896 |
| | | | | | | | (0.894-0.898) |
| CatBoost | 67 | 0.8511 | 0.7574 | 0.7805 | 0.8142 | 0.8046 | 0.892 |
| | | | | | | | (0.890-0.894) |
| H2O | 67 | 0.8770 | 0.7399 | 0.7735 | 0.8220 | 0.8089 | 0.897 |
| Ensemble | | | | | | | (0.894-0.899) |

Feature selection modeling

| Algorithm/ | | | | | | | |
|---|---|---|---|---|---|---|---|
| Modeling | | | | | | | |
| strategy | Feature | Sensitivity | Specificity | Precision | F1-score | Accuracy | AUROC |
| XGBoost | Top 45 | 0.8369 | 0.7789 | 0.7932 | 0.8145 | 0.8081 | 0.895 |
| | | | | | | | (0.892-0.897) |
| XGBoost | Top 35 | 0.8413 | 0.7735 | 0.7901 | 0.8149 | 0.8076 | 0.894 |
| | | | | | | | (0.892-0.897) |
| XGBoost | Top 25 | 0.8414 | 0.7706 | 0.7880 | 0.8138 | 0.8062 | 0.893 |
| | | | | | | | (0.891-0.896) |
| XGBoost | Top 10 | 0.8502 | 0.7496 | 0.7748 | 0.8108 | 0.8002 | 0.887 |
| | | | | | | | (0.885-0.890) |

As shown in Table 5, compared to the predictive sensitivity 76.1% of the multiple logistic regression model for the non-fasting status in the testing dataset, the XGBoost machine-learning model with full features shows a better sensitivity of 83.9, the accuracy of the XGBoost machine-learning model is 80.9%, and F1-score of the XGBoost machine-learning model is 81.6%.

Further, reference is made to Table 6, which shows the F-score values of the top 45 important features selected in XGBoost machine-learning model for the classification of fasting status and non-fasting status. As shown in Table 6, the residence-to-location distance, the age, the glucose, the height, and the level of serum creatinine are the top 5 important features for classification of fasting status and non-fasting status.

TABLE 6

| Order | Feature | F-score |
|---|---|---|
| 1 | Residence-to-location distance | 1863 |
| 2 | Age | 1234 |
| 3 | Glucose | 1024 |
| 4 | Height | 809 |
| 5 | SCr | 785 |
| 6 | LDL | 775 |
| 7 | Interval between request and sampling | 739 |
| 8 | Triglyceride | 681 |
| 9 | Body mass index | 618 |
| 10 | No. of outpatient visits | 617 |
| 11 | Total cholesterol | 603 |
| 12 | HDL | 603 |
| 13 | ALT | 591 |
| 14 | Weight | 543 |
| 15 | Uric acid | 499 |
| 16 | AST/ALT | 356 |
| 17 | BUN/SCr | 352 |
| 18 | Hemoglobin | 305 |
| 19 | Hemoglobin/Red blood cell count | 283 |
| 20 | Sampling timing of the day | 281 |
| 21 | AST | 263 |
| 22 | Serum potassium | 229 |
| 23 | Albumin | 214 |
| 24 | BUN | 207 |
| 25 | Serum sodium/serum potassium | 185 |
| 26 | Estimated blood osmolality | 177 |
| 27 | Red blood cell count | 177 |
| 28 | Specific gravity | 139 |
| 29 | Sulfonylurea | 116 |
| 30 | Sex | 115 |
| 31 | Serum sodium | 99 |
| 32 | Statins | 96 |
| 33 | Diabetes mellitus | 94 |
| 34 | Metabolism and Endocrinology | 87 |
| 35 | Metformin | 86 |
| 36 | Insulin | 86 |
| 37 | Stroke | 81 |
| 38 | Urine pH | 79 |
| 39 | General Medicine | 77 |
| 40 | Ticlopidine | 75 |
| 41 | Angiotensin-converting enzyme inhibitors | 72 |
| 42 | Thiazolinedione | 67 |
| 43 | Dipeptidyl peptidase-4 inhibitor | 66 |
| 44 | Beta blocker | 65 |
| 45 | Cardiology | 65 |

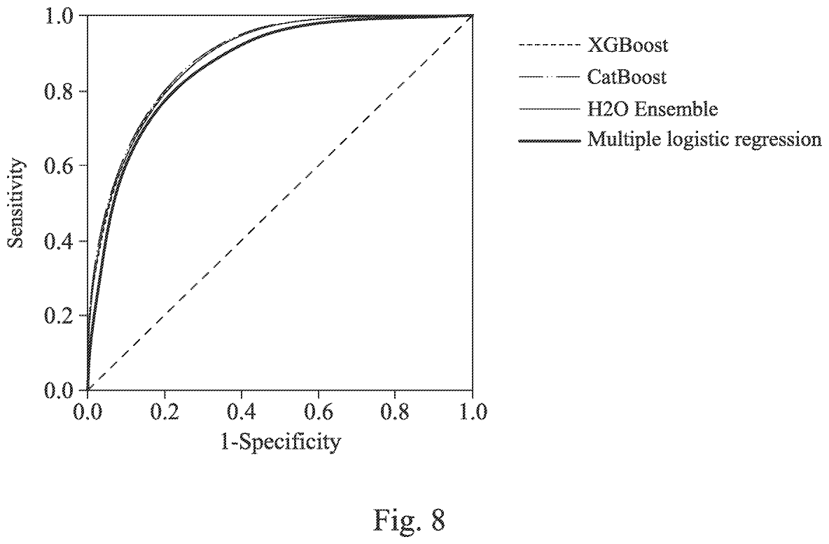

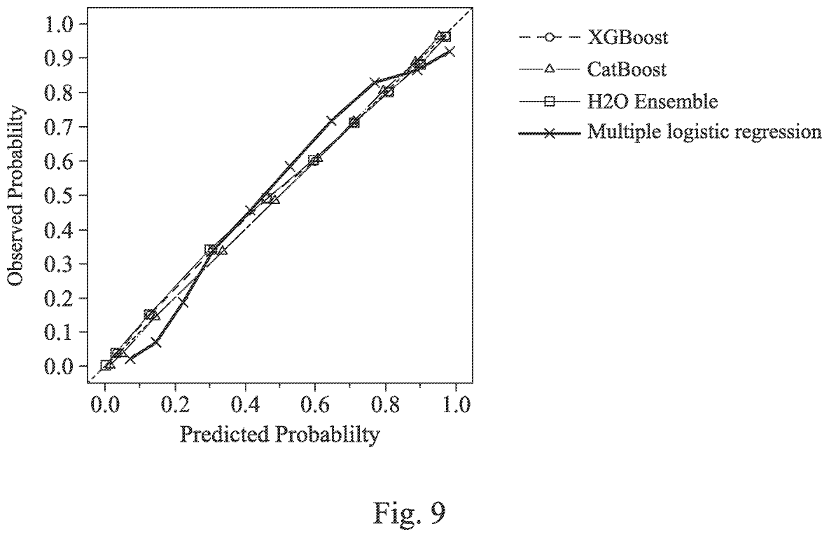

Reference is further made to FIG. 8 and FIG. 9, wherein FIG. 8 shows discrimination statistics results of different machine-learning models of the fasting status assessing system according to Example 1, and FIG. 9 shows calibration results of different machine-learning models of the fasting status assessing system according to Example 1.

As shown in FIG. 8 and FIG. 9, the AUROC and the calibration performance of the XGBoost machine-learning model, the CatBoost machine-learning model and the H2O AutoML ensemble machine-learning model are generally better than those of the multiple logistic regression model (AUROC 0.887 vs. 0.868, p<0.001).

As shown in the aforementioned results, the method for assessing fasting status and the fasting status assessing system of the present disclosure can reduce the proportion of ineffective glucose measurements, supporting their use in the real-world care flow to trigger actionable screening of diabetes mellitus. Further, the fasting-status assessing classifier also can help generate a warning upon detecting the discrepancy between the ontological fasting glucose level data and the non-fasting blood glucose data, which could serve as a checkpoint and a reminder in the automatically digital phenotyping process for diabetes mellitus screening. Hence, it is favorable for the diagnosis of the diabetes mellitus and designing the subsequent medical plans of the subject, and thus the method for assessing fasting status and the fasting status assessing system of the present disclosure has excellent clinical application potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A method for assessing fasting status, comprising:
providing a fasting blood glucose database, wherein the fasting blood glucose database comprises a plurality of fasting blood glucose data and a plurality of non-fasting blood glucose data;
performing a model establishing step, wherein the plurality of fasting blood glucose data and the plurality of non-fasting blood glucose data are trained to achieve a convergence by a machine-learning model so as to obtain a fasting-status assessing classifier;
providing an ontological data of a subject, wherein the ontological data comprises a blood glucose concentration data; and
performing an assessing step, wherein the ontological data is analyzed by the fasting-status assessing classifier to obtain an assessing result of fasting status of the subject.

2. The method of claim 1, wherein:
the fasting blood glucose database further comprises a plurality of ontological fasting glucose level data and a plurality of HbA1c-derived averaged glucose level data, each of the plurality of ontological fasting glucose level data is corresponding to one of the plurality of HbA1c-derived averaged glucose level data, and each of the plurality of ontological fasting glucose level data is compared to the HbA1c-derived averaged glucose level data corresponding thereto so as to obtain the plurality of fasting blood glucose data; and
each of the plurality of HbA1c-derived averaged glucose level data is obtained by calculating an HbA1c concentration data corresponding thereto with the following formula:
AG (mg/dL)=28.7×A1C−46.7,
wherein AG is the HbA1c-derived averaged glucose level data, and A1C is the HbA1c concentration data.

3. The method of claim 2, wherein when one of the plurality of ontological fasting glucose level data is not an ontological fasting glucose level data of a diabetic patient and is classified as the fasting blood glucose data, the one of the plurality of ontological fasting glucose level data satisfies the following conditions:
AC_{ontological}<100 mg/dL and A1C<5.5%; or
AC_{ontological}<AG−1 standard deviation of AC_{ontological};
wherein AC_{ontological} is the ontological fasting glucose level data, A1C is the HbA1c concentration data, and AG is the HbA1c-derived averaged glucose level data.

4. The method of claim 2, wherein when one of the plurality of ontological fasting glucose level data is an ontological fasting glucose level data of a diabetic patient and is classified as the fasting blood glucose data, the one of the plurality of ontological fasting glucose level data satisfies the following condition:
AC_{ontological}<AG;
wherein AC_{ontological} is the ontological fasting glucose level data, and AG is the HbA1c-derived averaged glucose level data.

5. The method of claim 1, wherein the machine learning algorithm module is a gradient descent algorithm.

6. The method of claim 5, wherein the gradient descent algorithm is an XGBoost machine-learning model, a CatBoost machine-learning model or an H2O AutoML ensemble machine-learning model.

7. The method of claim 1, wherein the ontological data further comprises a vital signs data, and the vital signs data comprises an age data, a gender data, a body mass index data and a medical history data.

8. The method of claim 7, wherein the ontological data further comprises a residence-to-location distance data, and the residence-to-location distance data is obtained by calculating a straight-line distance or a traffic distance between a residence of the subject and a fasting-status assessing location.

9. A fasting status assessing system, comprising:
a non-transitory machine readable medium for storing an ontological data of a subject, wherein the ontological data comprises a blood glucose concentration data; and
a processor signally connected to the non-transitory machine readable medium and comprising a fasting-status assessing classifier, wherein the ontological data is analyzed by the fasting-status assessing classifier to obtain an assessing result of fasting status of the subject.

10. The fasting status assessing system of claim 9, wherein the non-transitory machine readable medium is further for storing a fasting blood glucose database, and the fasting blood glucose database comprises a plurality of fasting blood glucose data and a plurality of non-fasting blood glucose data.

11. The fasting status assessing system of claim 10, wherein:
the fasting-status assessing classifier comprises a calculating module, the fasting blood glucose database further comprises a plurality of ontological fasting glucose level data and a plurality of HbA1c-derived averaged glucose level data, each of the plurality of ontological fasting glucose level data is corresponding to one of the plurality of HbA1c-derived averaged glucose level data, and each of the plurality of ontological fasting glucose level data is compared to the HbA1c-derived averaged glucose level data corresponding thereto by the calculating module so as to obtain the plurality of fasting blood glucose data; and
each of the plurality of HbA1c-derived averaged glucose level data is obtained by calculating an HbA1c concentration data corresponding thereto by the calculating module with the following formula:
AG (mg/dL)=28.7×A1C−46.7,
wherein AG is the HbA1c-derived averaged glucose level data, and A1C is the HbA1c concentration data.

12. The fasting status assessing system of claim 11, wherein when one of the plurality of ontological fasting glucose level data is not an ontological fasting glucose level data of a diabetic patient and is classified as the fasting blood glucose data, the one of the plurality of ontological fasting glucose level data satisfies the following conditions:
AC_{ontological}<100 mg/dL and A1C<5.5%; or
AC_{ontological}<AG−1 standard deviation of AC_{ontological};
wherein AC_{ontological} is the ontological fasting glucose level data, A1C is the HbA1c concentration data, and AG is the HbA1c-derived averaged glucose level data.

13. The fasting status assessing system of claim 11, wherein when one of the plurality of ontological fasting glucose level data is an ontological fasting glucose level data of a diabetic patient and is classified as the fasting blood glucose data, the one of the plurality of ontological fasting glucose level data satisfies the following condition:
AC_{ontological}<AG;
wherein AC_{ontological} is the ontological fasting glucose level data, and AG is the HbA1c-derived averaged glucose level data.

14. The fasting status assessing system of claim 10, wherein the fasting-status assessing classifier is obtained by training the plurality of fasting blood glucose data and the plurality of non-fasting blood glucose data to achieve a convergence by a machine-learning model.

15. The fasting status assessing system of claim 14, wherein the machine-learning model is a gradient descent algorithm.

16. The fasting status assessing system of claim 15, wherein the gradient descent algorithm is an XGBoost machine-learning model, a CatBoost machine-learning model or an H2O AutoML ensemble machine-learning model.

17. The fasting status assessing system of claim 9, wherein the ontological data further comprises a vital signs data, and the vital signs data comprises an age data, a gender data, a body mass index data and a medical history data.

18. The fasting status assessing system of claim 17, wherein the ontological data further comprises a residence-to-location distance data, and the residence-to-location distance data is obtained by calculating a straight-line distance or a traffic distance between a residence of the subject and a fasting-status assessing location.
